(19) United States
(12) Patent Application Publication
(10) Pub. No.: US 2018/0184900 A1
(43) Pub. Date: Jul. 5, 2018

(54) BLUETOOTH PULSE DIAGOSIS BRACELET AND METHOD FOR TRANSMITTING PULSE DATA

(71) Applicant: Shanghai University of Traditional Chinese Medicine, Shanghai (CN)

(72) Inventors: Jiatuo XU, Shanghai (CN); Ji CUI, Shanghai (CN); Liping TU, Shanghai (CN); Zhifeng ZHANG, Shanghai (CN); Yimin BAO, Shanghai (CN); Longtao CUI, Shanghai (CN); Jingbin HUANG, Shanghai (CN)

(21) Appl. No.: 15/309,032

(22) PCT Filed: Jul. 6, 2016
(86) PCT No.: PCT/CN2016/088926

§ 371 Date: Nov. 4, 2016

(30) Foreign Application Priority Data
May 6, 2016 (CN) 201610298414.6

(51) Int. Cl. A61B 5/00; A61B 5/024

(52) U.S. Cl. CPC A61B 5/0004; A61B 5/02438; A61B 5/4854; A61B 5/681; A61B 5/002

(57) ABSTRACT

The invention relates to the field of pulse diagnosis technology, more particularly, to a Bluetooth pulse diagnosis bracelet and a method for transmitting pulse data; the Bluetooth pulse diagnosis bracelet comprises: an acquisition unit and a Bluetooth transmission unit; the Bluetooth transmission unit further comprises: an information acquisition module and a transmission module; the method comprises: STEP S1, the Bluetooth pulse diagnosis bracelet monitoring the pulse data in real time; STEP S2, the Bluetooth pulse diagnosis bracelet acquiring the Bluetooth control information sent from the remote-connected intelligent terminal; STEP S3, the Bluetooth pulse diagnosis bracelet determining the meanings of the Bluetooth control information, and when the Bluetooth control information represents the communication is permitted, going to Step 4; STEP S4, the Bluetooth pulse diagnosis bracelet transmits the pulse data to the intelligent terminal according to the method of Bluetooth transmission.

### CROSS-REFERENCE TO RELATED APPLICATIONS

The present application claims priority to and the benefit of Chinese Patent Application No. CN 201610298414.6, filed on May 6, 2016, the entire content of which is incorporated herein by reference.

### BACKGROUND OF THE INVENTION

### 1. Field of the Invention

The invention relates to the field of pulse diagnosis technology, more specifically, to a Bluetooth pulse diagnosis bracelet and a method for transmitting pulse data.

### 2. Description of the Related Art

Pulse diagnosis is a method with Chinese medicine diagnosis characteristics, which has a unique theoretical system, and with the development and application of technology, pulse diagnosis has achieved great improvement in teaching, clinical medicine and science, and is being accepted by more and more people.

Pulse diagnosis depends on the pulse data of human body, and at present the pulse condition acquisition device is large and inconvenient for carrying, the data transmission of the acquisition device is adopted through cable transmission; for instance, by using USB or other cable to connect the computer for data acquisition and further analysis and so on. The acquisition method and data transmission method are limited by place and other factors; if the data cable itself has error, it would affect the acquisition of the pulse condition.

### SUMMARY OF THE INVENTION

Aimed at the above-mentioned problems existing in the prior art, this invention provides a Bluetooth pulse diagnosis bracelet and a method for transmitting pulse data, so as to make the pulse device easier and more convenient to wear, and make pulse data easier to transmit, and break through the length constraints of wire transmission.

The specific technical proposal is as follows:

a Bluetooth pulse diagnosis bracelet, wherein, comprises:

an acquisition unit, configured to acquire a wearer's pulse data in real time; and

a Bluetooth transmission unit, connected to the acquisition unit, and configured to transmit the pulse data to a remote-connected intelligent terminal through a method of Bluetooth transmission;

the Bluetooth transmission unit further comprises:

an information acquisition module, configured to acquire the Bluetooth control information sent from the intelligent terminal; and

a transmission module, connected to the information acquisition module, and configured to determine the Bluetooth control information, and send the pulse data to the intelligent terminal when the Bluetooth control information representing communication is permitted.

Preferably, the Bluetooth pulse diagnosis bracelet, wherein, the intelligent terminal is a mobile phone or a tablet.

Preferably, the Bluetooth pulse diagnosis bracelet, wherein, the Bluetooth control information is a characteristic value information of which UUID is 0Xff72;

The fixed length of the Bluetooth control information is 20 bytes.

Preferably, the Bluetooth pulse diagnosis bracelet, wherein the Bluetooth transmission unit comprises:

a feedback module, connected to the transmission module;

if the transmission module determines that the Bluetooth control information represents the communication is permitted, the feedback module will feedback a first feedback information to the intelligent terminal;

if the transmission module determines that the Bluetooth control information represents communication is not permitted, the feedback module will feedback a second feedback information to the intelligent terminal.

Preferably, the Bluetooth pulse diagnosis bracelet, wherein it further comprises:

a battery unit, configured to provide electric energy for the Bluetooth pulse diagnosis bracelet to work normally;

a power management unit, connected to the battery unit, and configured to monitor remaining capacity of the battery unit;

a feedback unit, connected to the power management unit and the Bluetooth transmission unit respectively, and configured to transmit the notification information, including the current remaining capacity, to the intelligent terminal through the method of Bluetooth transmission when the remaining battery capacity, got by monitoring, falls below a preset threshold; and the intelligent terminal notifies the wearer to replace battery according to the notification information.

Preferably, the Bluetooth pulse diagnosis bracelet, wherein it further comprises:

a battery unit, configured to provide electric energy for the Bluetooth pulse diagnosis bracelet;

a power management unit, connected to the battery unit, and configured to monitor remaining battery capacity of the battery unit;

a reading unit, connected to the power management unit and the Bluetooth transmission unit respectively, and configured to feedback the notification, including current remaining battery capacity, to the intelligent terminal through the method of Bluetooth transmission, so as to make the intelligent terminal to read the current remaining battery capacity of the battery unit.

Preferably, the Bluetooth pulse diagnosis bracelet, wherein, the notification information is a characteristic value information of which UUID is 0x2A19.

A method for transmitting the pulse data, adopting the Bluetooth pulse diagnosis bracelet, wherein, comprises:

STEP S1, the Bluetooth pulse diagnosis bracelet monitoring the pulse data in real time;

STEP S2, the Bluetooth pulse diagnosis bracelet acquiring the Bluetooth control information sent from the remote-connected intelligent terminal.

STEP S3, the Bluetooth pulse diagnosis bracelet determines the meanings of the Bluetooth control information, and when the Bluetooth control information representing that the communication is permitted, going to Step 4;

STEP S4, the Bluetooth pulse diagnosis bracelet transmits the pulse data to the intelligent terminal through the method of Bluetooth transmission.

The positive effects of the above-mentioned technical solution are:

1) A Bluetooth pulse diagnosis bracelet has been provided to make the pulse diagnosis device easier and more convenient to wear, make the pulse data easier to transmit, and break through length constraints of wire transmission.

2) A method for transmitting the pulse data is provided that is able to support the Bluetooth pulse diagnosis bracelet to run.

### BRIEF DESCRIPTIONS OF THE DRAWINGS

The accompanying drawings, together with the specification, illustrate exemplary embodiments of the present disclosure, and, together with the description, serve to explain the principles of the present invention.

FIG. 1 is a structure diagram of an embodiment of a Bluetooth pulse diagnosis bracelet.

FIG. 2 is a structure diagram of another embodiment of a Bluetooth pulse diagnosis bracelet.

FIG. 3 is a flow chart of the method can be applied in the Bluetooth pulse diagnosis bracelet for transmitting the pulse data.

### DETAILED DESCRIPTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “plurality” means a number greater than one.

Hereinafter, certain exemplary embodiments according to the present disclosure will be described with reference to the accompanying drawings.

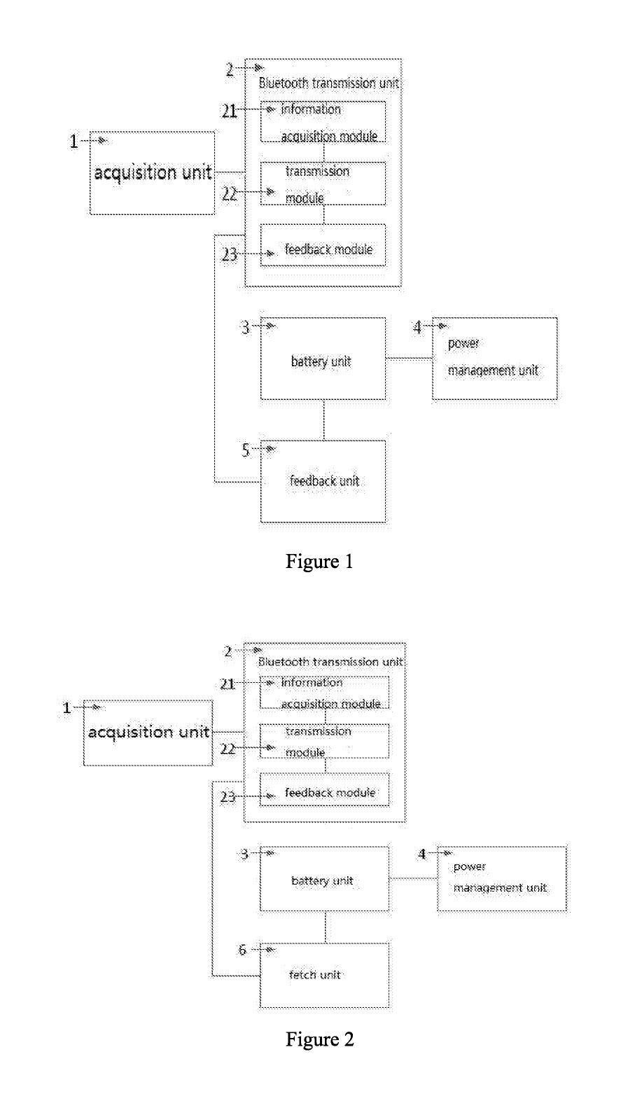

In a preferred embodiment, provide a Bluetooth pulse diagnosis bracelet, the bracelet comprising the structure in the following FIG. 1:

an acquisition unit 1, configured to acquire a wearer's pulse data in real time; and

a Bluetooth transmission unit 2, connected to the acquisition unit 1, and configured to transmit the pulse data to a remote-connected intelligent terminal through the method of Bluetooth transmission;

Still refer to FIG. 1, the Bluetooth transmission unit 2 further comprises:

an information acquisition module 21, configured to acquire the Bluetooth control information sent from the intelligent terminal; and

a transmission module 22, connected to the information acquisition module 21, and configured to determine the Bluetooth control information, and send the pulse data to the intelligent terminal when the Bluetooth control information representing communication is permitted; In an embodiment of the invention, the Bluetooth pulse diagnosis bracelet has a shape of a common intelligent bracelet, and it is internally set with: a acquisition unit 1, and the acquisition unit 1 is configured to acquire the user's pulse condition, so as to acquire the pulse data of the user in real time. The acquisition unit 1 connects to a Bluetooth transmission unit 2, and the Bluetooth transmission unit 2 is configured to transmit the pulse data acquired by the acquisition unit 1 to a remote-connected intelligent terminal (not shown) through the method of Bluetooth transmission.

In this embodiment of the invention, when Bluetooth transmission is required, the intelligent terminal will send a Bluetooth control information with a default format to the Bluetooth transmission unit 2 (the default format are described in detail below). The information acquisition module 21 in the Bluetooth transmission unit 2 acquires the Bluetooth control information, and use the above-mentioned transmission module 22 to determine whether the Bluetooth control information represents that the communication is permitted, and if the communication is permitted, use the transmission module 22 to send the real-time acquired pulse data to the intelligent device.

Specifically, the Bluetooth transmission unit 2 is operated in Bluetooth peripheral mode, and the upper-computer software in the intelligent device is operated in Bluetooth Central mode. The pulse diagnosis bracelet is in a broadcast state after powered on, and is not initiative to start a connection; the upper-computer software is to scan and start the connection, and after establishing a connection, communication parameters can be set by the Bluetooth transmission unit 2.

For instance, at the time when the intelligent terminal turn on the Bluetooth function configured on itself, the intelligent terminal establishes a Bluetooth connection with the Bluetooth pulse diagnosis bracelet, and then the intelligent terminal will open a specific pulse diagnosis application; at the time when opening the application, the intellectual terminal transmits the Bluetooth control information representing the communication is permitted to the Bluetooth pulse diagnosis bracelet; after the Bluetooth pulse diagnosis bracelet transmits the corresponding pulse data to the intelligent terminal after receiving the Bluetooth control information.

In a preferred embodiment of the invention, the above mentioed intelligent terminal can be a mobile phone or tablet. In other word, the intelligent terminal can be portable mobile devices, as to meet the portable characteristic of bracelet. The upper-computer software of the bracelet, such as pulse diagnosis application, can be installed on the mobile terminal; and when in use, the application is to be opened.

In a preferred embodiment of the invention, the Bluetooth control information is a characteristic value information of which UUID (Universally Unique Identifier) is 0Xff72, and the fixed length of the Bluetooth control information is 20 bytes.

Specifically, the above-mentioned Bluetooth control information can be expressed as DATA characteristic value, UUID of DATA is 0xFF72, the fixed length thereof is 20 bytes. When the upper-computer software of the intelligent terminal notifies the Bluetooth pulse diagnosis bracelet to transmit the data, the upper-computer software writes 0x0001 to the handle value of the DATA characteristic value, which is to add 1 to the handle value; thereafter, the Bluetooth pulse diagnosis bracelet transmits the data to the upper-computer software of the intelligent terminal through the method of Bluetooth transmission.

In a preferred embodiment of the invention, still referring to FIG. 1, the Bluetooth transmission unit 2 further comprises:

a feedback module 23, connected to the transmission module;

if the transmission module 22 determines that the Bluetooth control information represents the communication is permitted, the feedback module 23 will feedback a first feedback information to the intelligent terminal;

if the transmission module 22 determines that the Bluetooth control information represents the communication is not permitted, the feedback module 23 will feedback a second feedback information to the intelligent terminal.

Specifically, in a preferred embodiment of the invention, if the transmission module 22 determines the Bluetooth control information representing that the communication is permitted (the upper-computer software determines that the handle value of the DATA characteristic value is 0x0001), the feedback module 23 will feedback a first feedback information to the intelligent terminal, and the first feedback information can be a feedback information beginning with 0x20, and of which the fixed length is 20 bytes, i.e. 0x20000000000000000000, or a feedback information beginning with 0x32 and of which the fixed length is 20 bytes, i.e., 0x32000000000000000000.

Instead, in a preferred embodiment of the invention, if the transmission module 22 determine that the Bluetooth control information represents that the communication is not permitted, the feedback module 23 will feedback a second feedback information to the intelligent terminal, the second feedback information can be a feedback information beginning with 0x20 and of which the fixed length is 20 bytes, i.e. 0x20000000000000000000, or a feedback information that begins with 0x33 and of which the fixed length is 20 bytes, i.e. 0x33000000000000000000.

In a preferred embodiment of the invention, still referring to FIG. 1, the Bluetooth pulse diagnosis bracelet further comprises:

a battery unit 3, configured to provide electric energy for the Bluetooth pulse diagnosis bracelet to work normally;

a power management unit 4, connected to the battery unit 3, and configured to monitor the remaining capacity of the battery unit;

a feedback unit 5, connected to the power management unit 4 and the Bluetooth transmission unit 2 respectively, and configured to transmit the notification information, including the current remaining capacity, to the intelligent terminal through the method of Bluetooth transmission when the remaining battery capacity, acquired by monitoring falls below a preset threshold, and the intelligent terminal notifies the wearer to replace battery according to the notification information.

Specifically, in a preferred embodiment of the invention, the Bluetooth pulse diagnosis bracelet provides an interface service of which UUID=0x18F0, and the interface service is configured to provide an interface to read the remaining power, which is to set an interface to read the remaining battery capacity for the power management unit 4.

Thereafter, with the interface service a characteristic value of which UUID=0x2A19 is set, and the characteristic value can be read by the upper-computer software of the intelligent device. In this embodiment, the power management unit 4 can monitor the current remaining battery capacity of the battery unit through the interface service; thereafter through the feedback unit 5, when the current remaining battery capacity goes below the preset threshold value, the communication information will be sent to the intelligent terminal through the characteristic value of which UUID=0x2A19.

In a preferred embodiment of the invention, referring to the FIG. 2, the Bluetooth pulse diagnosis bracelet further comprises:

a battery unit 3, configured to provide electric energy for the Bluetooth pulse diagnosis bracelet;

a power management unit 4, connected to the battery unit 3, and configured to monitor the remaining battery capacity of the battery unit;

a reading unit 6, connected to the power management unit 4 and the Bluetooth transmission unit 2 respectively, and configured to feedback the notification, including current remaining battery capacity, to the intelligent terminal through the method of Bluetooth transmission, so as to make the intelligent terminal read the current remaining battery capacity of the battery unit.

Specifically, in a preferred embodiment of the invention, the Bluetooth pulse diagnosis bracelet provides an interface service of which UUID=0x18F0, and the interface service is configured to supply a interface to read the remaining power, and an interface to read the remaining battery capacity for the power management unit 4 is set.

Thereafter, with the interface service a characteristic value of which UUID=0x2A19 is set, and the characteristic value can be read by the upper-computer software of the intelligent device. In this embodiment, the upper-computer software of the intelligent device directly takes the characteristic value, and the current remaining battery capacity monitored by the power management unit 4 can be read through the reading unit 6, and when the remaining battery capacity falls below a preset threshold, the intelligent terminal will notify the wearer to replace battery.

In a preferred embodiment of the invention, the remaining battery capacity of the battery can be represented with percentage; for instance, for a Bluetooth pulse diagnosis bracelet of which the nominal voltage is 3V, take Q as the remaining battery capacity, thus:

If Q24 3V, it can represent 100% electric capacity;

If Q≤2V, it can represent 0% electric capacity;

If 2V<Q<3V, it can convert the remaining battery capacity into a corresponding percentage, such as 2.85V can be represented as 85% electric capacity.

In the preceding two embodiments of the invention, the preset threshold may be 10%, which is when 2V<Q≤2.1V, the intelligent device will notify the wearer to replace battery.

In a preferred embodiment of the invention, the above mentioned battery unit 3 further comprises a hardware dial switch (not shown), and the switch is equivalent to the main power switch of the Bluetooth pulse diagnosis bracelet, so as to control the battery unit 3 to supply power to each sensor module of the acquisition unit 1 and the Bluetooth transmission unit 2.

In a preferred embodiment of the invention, the above-mentioned battery unit 3 can still supply charging function, and the wearer can charge the battery unit 3 through a Micro-USB interface.

In conclusion, in the technical scheme of the invention, the invention provides a Bluetooth pulse diagnosis bracelet, which can acquire the wear's pulse data in real time, and transmit the pulse data to a remote-connected intelligent terminal through the method of Bluetooth transmission. The data transmission process between the intelligent terminal and the Bluetooth pulse diagnosis bracelet can be controlled by a customized Bluetooth transmission protocol, so as to make the pulse device easier and convenient to wear and portable.

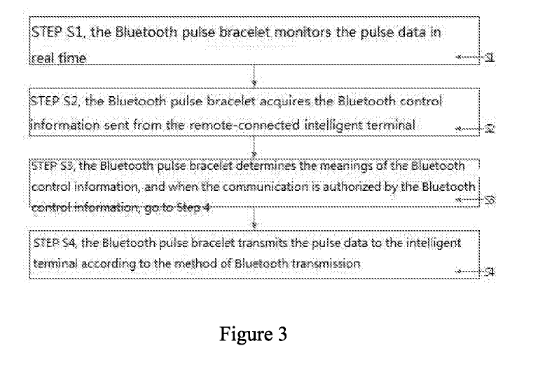

In an embodiment of the invention, base on the Bluetooth pulse diagnosis bracelet, further provide a method for transmitting the pulse data, the method is shown in FIG. 3, comprises:

STEP S1, the Bluetooth pulse diagnosis bracelet monitoring wear's pulse data in real time;

STEP S2, the Bluetooth pulse diagnosis bracelet acquiring the Bluetooth control information sent from the remote-connected intelligent terminal;

STEP S3, the Bluetooth pulse diagnosis bracelet determining the meanings of the Bluetooth control information, and when the Bluetooth control information representing that the communication is permitted, going to Step 4;

STEP S4, the Bluetooth pulse diagnosis bracelet transmitting the pulse data to the intelligent terminal through the method of Bluetooth transmission.

The foregoing is only the preferred embodiments of the invention, not thus limiting embodiments and scope of the invention, those skilled in the art should be able to realize that the schemes obtained from the content of specification and figures of the invention are within the scope of the invention.

What is claimed is:

1. A Bluetooth pulse diagnosis bracelet, comprising:
an acquisition unit, configured to acquire a wearer's pulse data in real time; and
a Bluetooth transmission unit, connected to the acquisition unit, and configured to transmit the pulse data to a remote-connected intelligent terminal through a method of Bluetooth transmission;
the Bluetooth transmission unit further comprises:
an information acquisition module, configured to acquire Bluetooth control information sent from the intelligent terminal; and
an transmission module, connected to the information acquisition module, and configured to determine the Bluetooth control information, and send the pulse data to the intelligent terminal when the Bluetooth control information representing communication is permitted.

2. The Bluetooth pulse diagnosis bracelet according to claim 1, wherein the intelligent terminal is a mobile phone or a tablet.

3. The Bluetooth pulse diagnosis bracelet according to claim 1, wherein the Bluetooth control information is a characteristic value information of which UUID is 0xFF72.

4. The Bluetooth pulse diagnosis bracelet according to claim 3, wherein the Bluetooth transmission unit comprises:
a feedback module, connected to the transmission module;
if the transmission module determines that the Bluetooth control information represents the communication is permitted, the feedback module will feedback a first feedback information to the intelligent terminal;
if the transmission module determines that the Bluetooth control information represents the communication is not permitted, the feedback module will feedback a second feedback information to the intelligent terminal.

5. The Bluetooth pulse diagnosis bracelet according to claim 3, further comprises:
a battery unit, configured to provide electric energy for the Bluetooth pulse diagnosis bracelet to work normally;
a power management unit, connected to the battery unit, and configured to monitor remaining battery capacity of the battery unit;
a feedback unit, connected to the power management unit and the Bluetooth transmission unit respectively, and configured to transmit notification information, including the current remaining capacity, to the intelligent terminal through the method of Bluetooth transmission when the remaining battery capacity, got by monitoring, falls below a preset threshold; and the intelligent terminal notifies the wearer to replace battery according to the notification information.

6. The Bluetooth pulse diagnosis bracelet according to claim 3, further comprises:
a battery unit, configured to provide electric energy for the Bluetooth pulse diagnosis bracelet;
a power management unit, connected to the battery unit, and configured to monitor remaining battery capacity of the battery unit;
a reading unit, connected to the power management unit and the Bluetooth transmission unit respectively, and configured to feedback the notification, including current remaining battery capacity, to the intelligent terminal through the method of Bluetooth transmission, so as to make the intelligent terminal read the current remaining battery capacity of the battery unit.

7. The Bluetooth pulse diagnosis bracelet according to claim 5, the notification information is a characteristic value information of which UUID is 0x2A19.

8. The Bluetooth pulse diagnosis bracelet according to claim 6, the notification information is a characteristic value information of which UUID is 0x2A19.

9. The method for transmitting pulse data, adopting a Bluetooth pulse diagnosis bracelet, the Bluetooth pulse diagnosis bracelet comprising:
an acquisition unit, configured to acquire a wearer's pulse data in real time; and
a Bluetooth transmission unit, connected to the acquisition unit, and configured to transmit the pulse data to a remote-connected intelligent terminal through a method of Bluetooth transmission;
the Bluetooth transmission unit further comprises:
an information acquisition module, configured to acquire Bluetooth control information sent from the intelligent terminal; and
an transmission module, connected to the information acquisition module, and configured to determine the Bluetooth control information, and send the pulse data to the intelligent terminal when the Bluetooth control information representing communication is permitted;
the method comprising:
STEP S1, the Bluetooth pulse diagnosis bracelet monitoring wear's pulse data in real time;
STEP S2, the Bluetooth pulse diagnosis bracelet acquiring the Bluetooth control information sent from the remote-connected intelligent terminal.
STEP S3, the Bluetooth pulse diagnosis bracelet determining the meanings of the Bluetooth control information, and when the Bluetooth control information representing that the communication is permitted, going to Step 4;
STEP S4, the Bluetooth pulse diagnosis bracelet transmitting the pulse data to the intelligent terminal through the method of Bluetooth transmission.

10. The method for transmitting pulse data according to claim 9, wherein the intelligent terminal is a mobile phone or a tablet.

11. The method for transmitting pulse data according to claim 9, wherein the Bluetooth control information is a characteristic value information of which UUID is 0xFF72.

12. The method for transmitting pulse data according to claim 11, wherein the Bluetooth transmission unit comprises:
a feedback module, connected to the transmission module;
if the transmission module determines that the Bluetooth control information represents the communication is permitted, the feedback module will feedback a first feedback information to the intelligent terminal;
if the transmission module determines that the Bluetooth control information represents the communication is not permitted, the feedback module will feedback a second feedback information to the intelligent terminal.

13. The method for transmitting pulse data according to claim 11, wherein, the Bluetooth pulse diagnosis bracelet further comprises:
a battery unit, configured to provide electric energy for the Bluetooth pulse diagnosis bracelet to work normally;
a power management unit, connected to the battery unit, and configured to monitor remaining battery capacity of the battery unit;
a feedback unit, connected to the power management unit and the Bluetooth transmission unit respectively, and configured to transmit notification information, including the current remaining capacity, to the intelligent terminal through the method of Bluetooth transmission when the remaining battery capacity, got by monitoring, falls below a preset threshold; and the intelligent terminal notifies the wearer to replace battery according to the notification information.

14. The method for transmitting pulse data according to claim 11, wherein, the Bluetooth pulse diagnosis bracelet further comprises:
a battery unit, configured to provide electric energy for the Bluetooth pulse diagnosis bracelet;
a power management unit, connected to the battery unit, and configured to monitor remaining battery capacity of the battery unit;
a reading unit, connected to the power management unit and the Bluetooth transmission unit respectively, and configured to feedback the notification, including current remaining battery capacity, to the intelligent terminal through the method of Bluetooth transmission, so as to make the intelligent terminal read the current remaining battery capacity of the battery unit.

15. The method for transmitting pulse data according to claim 13, the notification information is a characteristic value information of which UUID is 0x2A19.

16. The method for transmitting pulse data according to claim 14, the notification information is a characteristic value information of which UUID is 0x2A19.
